(19) United States
(12) Patent Application Publication
(10) Pub. No.: US 2022/0401567 A1
(43) Pub. Date: Dec. 22, 2022

(54) USES OF HYALURONAN CONJUGATE

(71) Applicant: Aihol Corporation, Buena Park, CA (US)

(72) Inventor: Hua-Yang LIN, Taipei City (TW)

(73) Assignee: Aihol Corporation, Buena Park, CA (US)

(21) Appl. No.: 17/843,534

(22) Filed: Jun. 17, 2022

Related U.S. Application Data
(60) Provisional application No. 63/212,297, filed on Jun. 18, 2021.

(51) Int. Cl. A61K 47/61

(52) U.S. Cl. CPC A61K 47/61

(57) ABSTRACT

Disclosed herein is the use of a hyaluronan (HA) conjugate for treating cancer. Also disclosed herein is the use of a hyaluronan conjugate for treating cancer. The hyaluronan conjugate is a nimesulide-HA conjugate having a monosaccharide or one to four disaccharide units of the hyaluronic acid.

### BACKGROUND OF THE INVENTION

### 1. Field of the Invention

The present disclosure relates to hyaluronic conjugates; more particularly, to hyaluronic conjugates for treating cancer.

### 2. Description of Related Art

Cancer is among the leading causes of death worldwide, accounting for nearly 10 million deaths in 2020. According to World Health Organization's statistics, the five most common new cases of cancer in 2020 were: breast cancer (2.26 million cases), lung cancer (2.21 million cases), colon and rectum cancer (1.93 million cases), prostate cancer (1.41 million cases), and skin cancer (non-melanoma) (1.20 million cases). The most common causes of cancer death in 2020 were: lung cancer (1.80 million deaths), colon and rectum cancer (935,000 deaths), liver cancer (830,000 deaths), stomach cancer (769,000 deaths), and breast cancer (685,000 deaths).

Currently, there are four standard methods of treatment for cancer: radiotherapy, chemotherapy, surgery therapy, and/or immunotherapy; among them, surgery and chemotherapy remain the most adopted means for cancer treatment. Determining the goals of treatment is an important first step. The primary goal is generally to cure cancer or to considerably prolong life. In many cases, surgery is used with the aim of complete removal of the cancer without significantly damage to the rest of the body, but the propensity of cancers to invade adjacent tissue or to spread to distant sites by microscopic metastasis often limits its effectiveness. Chemotherapy is the treatment of cancer with cytotoxic drugs that can destroy cancer cells. However, in some occasions, cancerous cells develop resistance or are insensitive toward one or more cytotoxic drugs, thereby rendering these drug(s) useless for the treatment of cancer. Further, some cancers (such as lung cancer) have poor prognosis or are insensitive to current chemotherapeutic agents. Hence, many attempts have been made to locate other effective drugs or means for successful treatment of cancer, especially drug resistant- or chemotherapeutic agent insensitive-cancer.

Therefore, there is a need in the related art for an agent or a compound that may be useful for the treatment of cancers.

### SUMMARY

The following presents a simplified summary of the disclosure in order to provide a basic understanding to the reader. This summary is not an extensive overview of the disclosure, and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method for treating cancer in a subject in need thereof.

In some embodiments, the method comprises the step of administering to the subject an effective amount of a hyaluronan conjugate, wherein the hyaluronan conjugate comprises a hyaluronan moiety of (−4GlcUAβ1-3GlcNAcβ1−)ₙ, where n=1-4, and the substituent groups (e.g., a cytotoxic drug) can be attached to one or more of the carboxyl groups of the D-glucuronic acid (4GlcUA).

In some other embodiments, the hyaluronan conjugate comprises a hyaluronan moiety of 3GlcNAcβ1-(4GlcUAβ1-3GlcNAcβ1−), or (4GlcUAβ1-3GlcNAcβ1−)ₙ-4GlcUAβ1, where n=1-4, and the substituent groups (e.g., a cytotoxic drug) can be attached to one or more of the carboxyl groups of the D-glucuronic acid (4GlcUA).

According to some embodiments of the present disclosure, the method comprises the step of administering to the subject an effective amount of a hyaluronan conjugate, wherein the hyaluronan conjugate comprises only two disaccharide units and has the structure of,

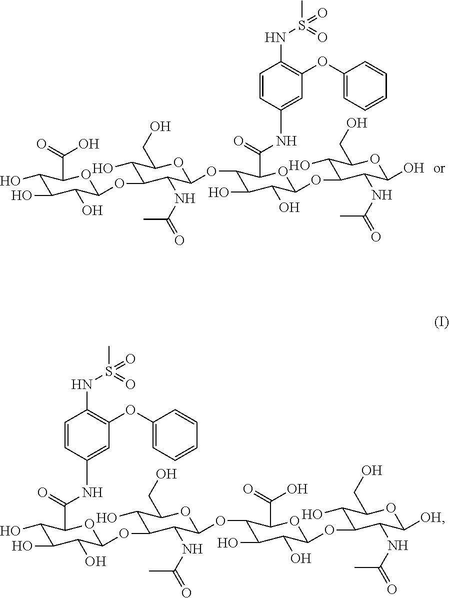 [SMILES: CC(=O)NC1C(O)OC(CO)C(O)C1OC1OC(C(=O)Nc2ccc(NS(C)(=O)=O)c(Oc3ccccc3)c2)C(OC2OC(CO)C(O)C(OC3OC(C(=O)O)C(O)C(O)C3O)C2NC(C)=O)C(O)C1O ; CC(=O)NC1C(O)OC(CO)C(O)C1OC1OC(C(=O)O)C(OC2OC(CO)C(O)C(OC3OC(C(=O)Nc4ccc(NS(C)(=O)=O)c(Oc5ccccc5)c4)C(O)C(O)C3O)C2NC(C)=O)C(O)C1O]

or a pharmaceutically acceptable salt thereof.

In some embodiments, the hyaluronan conjugate has the structure of,

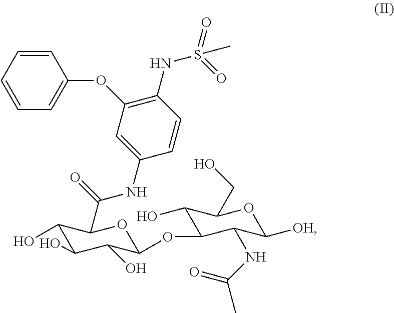 [SMILES: CC(=O)NC1C(O)OC(CO)C(O)C1OC1OC(C(=O)Nc2ccc(NS(C)(=O)=O)c(Oc3ccccc3)c2)C(O)C(O)C1O]

or a pharmaceutically acceptable salt thereof.

In some embodiments, the hyaluronan conjugate has the structure of,

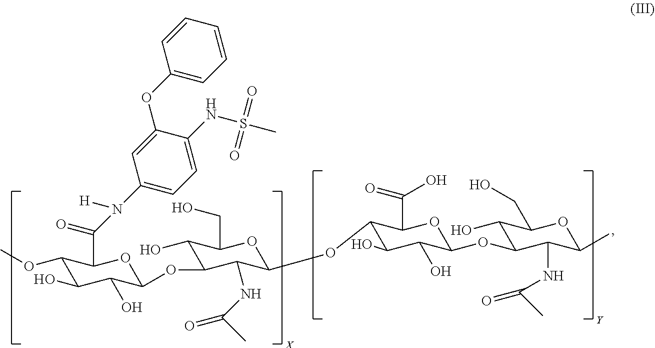

or a pharmaceutically acceptable salt thereof, where X is 1 and Y is 0, 1, 2, or 3.

In various embodiments, the subject is a mammal, including humans.

According to some embodiments of the present disclosure, the cancer is breast cancer, lung cancer, or colorectal cancer. For example, lung cancer can be non-small cell lung cancer (e.g., squamous cell carcinoma, adenocarcinoma, and large cell carcinoma) or small cell lung cancer.

In some embodiments, the hyaluronan conjugate is administered via intravenous (i.v.) injection.

In still another aspect, the present disclosure is directed to a pharmaceutical composition for treating cancer.

According to some embodiments, the pharmaceutical composition comprises an effective amount of a hyaluronic conjugate as described above and a pharmaceutically-acceptable excipient.

Subject matters that are also included in other aspects of the present disclosure include the use of a hyaluronic conjugate in the manufacture of a medicament for use in the treatment of cancer, as well as a hyaluronic conjugate for use in the treatment of cancer.

Many of the present disclosure's attendant features and advantages will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a line graph showing the tumor cell cytotoxicity of HA conjugates to A549 cell line (*:p<0.05 vs. Nim; #:p<0.05 vs. HA4).

FIG. 2 is a line graph showing the tumor cell cytotoxicity of HA conjugates to MDA-MB-231 cell line (*:p<0.05 vs. Nim; #:p<0.05 vs. HA4).

FIG. 3 is a line graph showing the tumor cell cytotoxicity of HA conjugates to HT-29 cell line (*:p<0.05 vs. Nim; #:p<0.05 vs. HA4).

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples, and appended claims are collected here. Unless otherwise defined herein, scientific and technical terminologies employed in the present disclosure shall have meanings commonly understood and used by one of ordinary skill in the art.

Unless otherwise required by context, it will be understood that singular terms shall include plural forms of the same, and plural terms shall include the singular. Also, as used herein and in the claims, the terms “at least one” and “one or more” have the same meaning and include one, two, three, or more. Furthermore, the phrases “at least one of A, B, and C,” “at least one of A, B, or C,” and “at least one of A, B, and/or C” as used throughout this specification and the appended claims, are intended to cover A alone, B alone, C alone, A and B together, B and C together, A and C together, as well as A, B, and C together.

Notwithstanding that the numerical ranges and parameters setting forth the broad scope of the invention are approximations, the numerical values outlined in the specific examples are reported as precisely as possible. However, any numerical value inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values, and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints unless specified otherwise.

The terms “treatment” and “treating” as used herein may refer to a preventative (e.g., prophylactic), curative or palliative measure. In particular, the term “treating” as used herein refers to the application or administration of the present hyaluronan conjugate or a pharmaceutical composition comprising the same to a subject, who has a medical condition, a symptom associated with the medical condition, a disease or disorder secondary to the medical condition, or a predisposition toward the medical condition, with the purpose to partially or wholly alleviate, ameliorate, relieve, delay the onset of, inhibit the progression of, reduce the severity of, and/or reduce the incidence of one or more symptoms or features of said particular disease, disorder, and/or condition. Treatment may be administered to a subject who does not exhibit signs of a disease, disorder, and/or condition, and/or to a subject who exhibits only early signs of a disease, disorder and/or condition, for the purpose of decreasing the risk of developing pathology associated with the disease, disorder and/or condition.

The terms “subject” and “patient” are used interchangeably herein and are intended to mean an animal including the human species that is treatable by the hyaluronan conjugate described herein, pharmaceutical compositions comprising the same, and/or methods of the present invention. Accordingly, the term “subject” or “patient” comprises any mammal, which may benefit from the present disclosure. The term “mammal” refers to all members of the class Mammalia, including humans, primates, domestic and farm animals, such as rabbit, pig, sheep, and cattle; as well as zoo, sports, or pet animals; and rodents, such as mouse and rat. The term “non-human mammal” refers to all members of the class Mammalia except humans. In one exemplary embodiment, the patient is a human. The term “subject” or “patient” are intended to refer to both the male and female gender unless one gender is specifically indicated.

The terms “application” and “administration” are used interchangeably herein to mean the application of a hyaluronan conjugate or a pharmaceutical composition of the present invention to a subject in need of treatment thereof.

The term “effective amount,” as used herein, refers to the quantity of the present hyaluronan conjugate that is sufficient to yield a desired therapeutic response. An effective amount of an agent is not required to cure a disease or condition but will provide treatment for a disease or condition such that the onset of the disease or condition is delayed, hindered, or prevented, or the disease or condition symptoms are ameliorated. The effective amount may be divided into one, two, or more doses in a suitable form to be administered at one, two, or more times throughout a designated period. The specific effective or sufficient amount will vary with such factors as the particular condition being treated, the physical condition of the patient (e.g., the ‘patient's body mass, age, or gender), the type of mammal or animal being treated, the duration of the treatment, the nature of concurrent therapy (if any), and the specific formulations employed and the structure of the compounds or its derivatives. The effective amount may be expressed, for example, as the total mass of the hyaluronan conjugate or the equivalent mass of the 4-aminonimesulide in the hyaluronan conjugate (e.g., in grams, milligrams, or micrograms) or a ratio of the mass of the hyaluronan conjugate or the equivalent mass of the 4-aminonimesulide in the hyaluronan conjugate to body mass, e.g., as milligrams per kilogram (mg/kg).

Also, according to the examples provided hereinbelow, the hyaluronan conjugate is administered via i.v. injection; however, this is only an illustration as to how the present invention can be implemented, and the present disclosure is not limited thereto.

For example, the hyaluronan conjugate can be formulated, together with a pharmaceutically-acceptable excipient, into a pharmaceutical composition suitable for the desired administration mode. Certain pharmaceutical compositions prepared in accordance with the presently disclosed and claimed inventive concept(s) are single unit dosage forms suitable for oral, mucosal (e.g., nasal, sublingual, vaginal, buccal, or rectal), parenteral (e.g., subcutaneous, intravenous, bolus injection, intramuscular, or intraarterial), intravitreal, or transdermal administration to a patient. Examples of dosage forms include, but are not limited to, tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient. As could be appreciated, these pharmaceutical compositions are also within the scope of the present disclosure.

The phrase “pharmaceutically acceptable excipient” as used herein means a pharmaceutically acceptable material, composition, or vehicle, such as a liquid or solid filler, diluent, carrier, solvent, or encapsulating material, involved in carrying or transporting the subject agents from one organ, or portion of the body, to another organ, or portion of the body. Each excipient must be “acceptable” in the sense of being compatible with the other ingredients of the formulation. The pharmaceutical formulation contains a compound of the invention in combination with one or more pharmaceutically acceptable ingredients. The excipient can be in the form of a solid, semi-solid or liquid diluent, cream, or capsule. These pharmaceutical preparations are a further object of the invention. Usually, the amount of active compounds is between 0.1-95% by weight of the preparation, preferably between 0.2-20% by weight in preparations for parenteral use, and preferably between 1 and 50% by weight in preparations for oral administration. For the clinical use of the methods of the present invention, the pharmaceutical composition of the invention is formulated into formulations suitable for the intended route of administration.

As used herein, the term “hyaluronic acid” (HA) (also called hyaluronate or hyaluronan) is an anionic, nonsulfated glycosaminoglycan composed of at least one disaccharide unit, specifically a D-glucuronic acid and an N-acetyl-D-glucosamine (−4GlcUAβ1-3GlcNAcβ1−). In some embodiments of the present disclosure, the HA consists of two disaccharide units (i.e., HA tetrasaccharide). The term “HA derivative” refers to an HA having any modification on the hydroxyl, carboxyl, amide, or acetylamino groups of one or more disaccharide units of the HA. According to certain embodiments of the present disclosure, the HA conjugates are in the form of a metal salt, preferably an alkali metal salt, and more preferably a sodium or potassium salt.

According to certain embodiments of the present disclosure, the hyaluronan conjugate comprises a hyaluronan moiety of (−4GlcUAβ1-3GlcNAcβ1-)ₙ, where n=1-4, and the substituent groups (i.e., a cytotoxic drug) can be attached to one or more of the carboxyl groups of the D-glucuronic acid (4GlcUA). For example, the cytotoxic drug is hydrogenated nimesulide.

In some other embodiments, the hyaluronan conjugate comprises a hyaluronan moiety of 3GlcNAcβ1-(4GlcUAβ1-3GlcNAcβ1−), or (4GlcUAβ1-3GlcNAcβ1−)ₙ−4GlcUAβ1, where n=1-4, and the substituent groups (i.e., a cytotoxic drug) can be attached to one or more of the carboxyl groups of the D-glucuronic acid (4GlcUA). For example, the cytotoxic drug is hydrogenated nimesulide.

Specifically, in certain embodiments, the hyaluronan conjugate comprises only two disaccharide units and has the structure of,

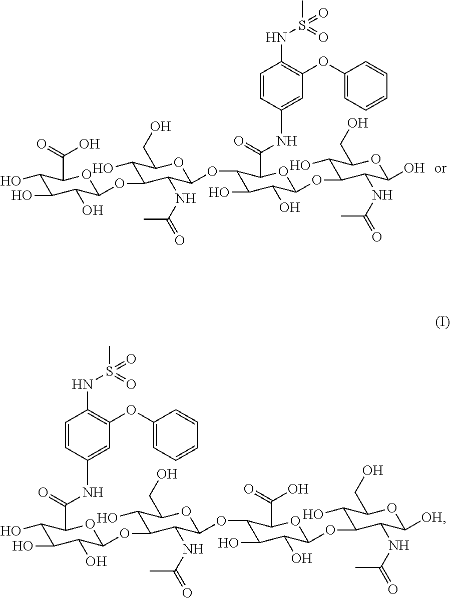 [SMILES: CC(=O)NC1C(O)OC(CO)C(O)C1OC1OC(C(=O)Nc2ccc(NS(C)(=O)=O)c(Oc3ccccc3)c2)C(OC2OC(CO)C(O)C(OC3OC(C(=O)O)C(O)C(O)C3O)C2NC(C)=O)C(O)C1O ; CC(=O)NC1C(O)OC(CO)C(O)C1OC1OC(C(=O)O)C(OC2OC(CO)C(O)C(OC3OC(C(=O)Nc4ccc(NS(C)(=O)=O)c(Oc5ccccc5)c4)C(O)C(O)C3O)C2NC(C)=O)C(O)C1O]

or a pharmaceutically acceptable salt thereof.

In some embodiments, the hyaluronan conjugate has the structure of,

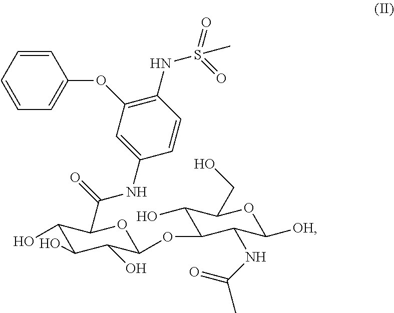 [SMILES: CC(=O)NC1C(O)OC(CO)C(O)C1OC1OC(C(=O)Nc2ccc(NS(C)(=O)=O)c(Oc3ccccc3)c2)C(O)C(O)C1O]

or a pharmaceutically acceptable salt thereof.

In some embodiments, the hyaluronan conjugate has the structure of,

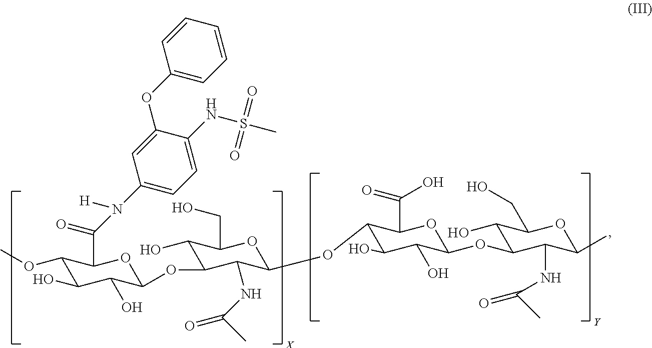

or a pharmaceutically acceptable salt thereof, where X is 1 and Y is 0, 1, 2, or 3.

As could be appreciated, when the hyaluronan moiety has more than one carboxyl group, “degree of substitution (DS)” of the HA conjugate (i.e., the ratio of the substituent groups (i.e., the hydrogenated nimesulide) attached per carboxyl group of the HA moiety) may be 20 to 100%. Specifically, for an HA conjugate having a hyaluronan moiety of (−4GlcUAβ1-3GlcNAcβ1−)₄, if the HA conjugate has 1, 2, 3, or 4 substituent groups, then the DS would be 25, 50, 75, or 100%, respectively. In another example, for an HA conjugate having a hyaluronan moiety of (4GlcUAβ1-3GlcNAcβ1−)₄-4GlcUAβ1, if the HA conjugate has 1, 2, 3, 4, or 5 substituent groups, then the DS would be 20, 40, 60, 80, or 100%, respectively.

The present disclosure is based, at least in part, on the discovery that the nimesulide-HA tetrasaccharide conjugate (NIM-Tetra) exhibits unexpected better tumor cell cytotoxicity to certain cancer cell lines, compared with nimesulide alone or other nimesulide-HA conjugates.

In view of the foregoing, the present disclosure proposes methods for treating cancer, particularly breast cancer, lung cancer, and colorectal cancer. Also provided herein is the use of said hyaluronan conjugate in the treatment of cancer, as well as its use in the manufacture of a medicament for said treatment purpose. The medicament (i.e., a pharmaceutical composition comprising the hyaluronan conjugate) is, of course, a subject matter covered by the scope of the present application.

The following Examples are provided to elucidate certain aspects of the present invention and aid those skilled in the art in practicing this invention. These Examples are in no way to be considered to limit the scope of the invention in any manner. Without further elaboration, it is believed that one skilled in the art can, based on the description herein, utilize the present invention to its fullest extent.

### Example 1

Synthesis and Characterization of NIM-HA Conjugates

(1) Preparation of Hydrogenated Nimesulide (H-NIM)

Hydrogenated nimesulide (N-(4-amino-2-phenoxyphenyl)methanesulfonamide, or 4-aminonimesulide) was synthesized from commercially purchased nimesulide (N-(4-nitro-2-phenoxyphenyl)methanesulfonamide). Briefly, 500 mg of nimesulide was completely dissolved in 20 ml ethyl acetate, and then 200 mg of 5% Pd/C (palladium on carbon) as catalyst was added into the solution. The air was extracted from the bottle under continued stir, and the air was replaced by hydrogen gas of up to 1 atm., followed by stirring for 24 hours to obtain the hydrogenated nimesulide (H-NIM).

The purity of the H-NIM was determined using thin-layer chromatography (TLC) on pre-coated TLC plates with silica gel 60 F254 under 254 nm UV light; mobile phase: hexane:ethyl acetate=2:1.

The Pd/C catalyst was removed by filtration, and the filtrate was concentrated on a rotary evaporator to remove the residual solvent. The hydrogenated product was then dissolved in hexane-ethyl acetate (1:1) solution for further purification on a silica gel column. The column was eluted with the elution solution (hexane-ethyl acetate=1:1). The fraction with color was collected and freeze-dried. The concentration and the structure of the resultant product were confirmed using UV and NMR, respectively.

(2) Synthesis of NIM-HA Tetrasaccharide Conjugate

HA tetrasaccharide (HA4) having two disaccharide units of D-glucuronic acid and N-acetylglucosamine was reacted with 4-aminonimesulide (hydrogenated nimesulide, H-NIM) to give NIM-HA tetrasaccharide conjugate (NIM-Tetra). Briefly, 50 mg of HA4 (0.06437 mmol) was dissolved in 3 mL DDW; then, 5 mL DMSO was added to the HA4 solution. 9.1 mg of Oxyma (0.06437 mmol) was dissolved in 0.5 mL DMSO and then added to the HA4 solution under stirring for 6 minutes; then, the 17.9 mg of H-NIM (0.07081 mmol) was dissolved in 0.5 mL DMSO and added to the HA4 solution. 14 μL DIC (0.09656 mmol) was dissolved in 0.5 mL DMSO under stirring for 1 minute and then added to the HA4 solution. The final reaction mixture was stirred for 48 hours at room temperature, and the resulting solution was freeze dryer for three days, and the separation and purification were achieved by preparative HPLC (Waters 600) with Insertsil ODS-3 column. The thus-synthesized NIM-Tetra has the following structures:

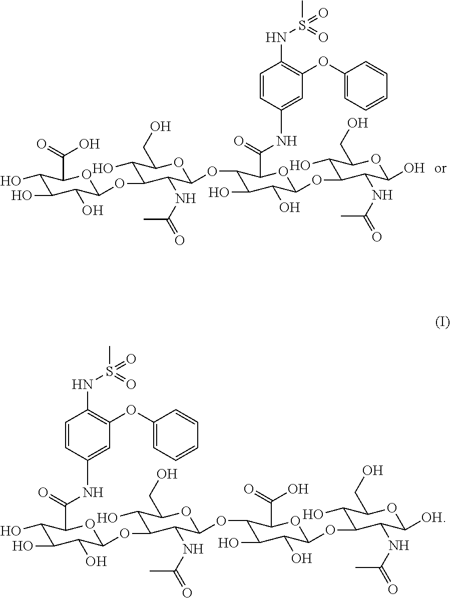 [SMILES: CC(=O)NC1C(O)OC(CO)C(O)C1OC1OC(C(=O)Nc2ccc(NS(C)(=O)=O)c(Oc3ccccc3)c2)C(OC2OC(CO)C(O)C(OC3OC(C(=O)O)C(O)C(O)C3O)C2NC(C)=O)C(O)C1O ; CC(=O)NC1C(O)OC(CO)C(O)C1OC1OC(C(=O)O)C(OC2OC(CO)C(O)C(OC3OC(C(=O)Nc4ccc(NS(C)(=O)=O)c(Oc5ccccc5)c4)C(O)C(O)C3O)C2NC(C)=O)C(O)C1O]

(3) Synthesis of NIM-HA Disaccharide Conjugate

HA disaccharide having one disaccharide unit of D-glucuronic acid and N-acetylglucosamine was reacted with 4-aminonimesulide (hydrogenated nimesulide, H-NIM) to give NIM-HA disaccharide (NIM-Di) conjugate having the following structure:

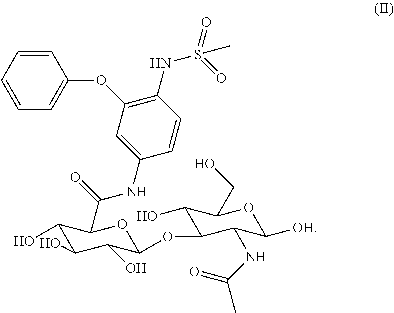 [SMILES: CC(=O)NC1C(O)OC(CO)C(O)C1OC1OC(C(=O)Nc2ccc(NS(C)(=O)=O)c(Oc3ccccc3)c2)C(O)C(O)C1O]

(4) Synthesis of NIM-HA Monosaccharide Conjugate

Briefly, glucuronic acid (194.14 Da) was reacted with 4-aminonimesulide (hydrogenated nimesulide, H-NIM) to give NIM-HA monosaccharide (454.4 Da) (NIM-Mono) conjugate having the following structure:

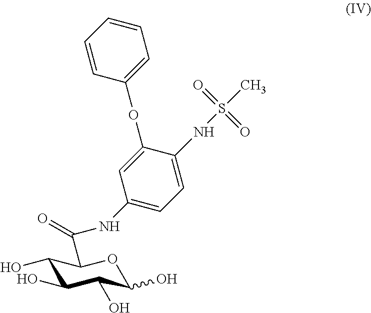

### Example 2

Tumor Cell Cytotoxicity of NIM-HA Conjugates to MDA-MB-231 Cells

In this example, MDA-MB-231 cells (human breast cancer cell line) were used to assess the tumor cell cytotoxicity of various NIM-HA conjugates.

MDA-MB-231 cells in 100 μL L-15 medium were seeded in a density of 5×10³ per well in a 96-well plate and incubated overnight. The next day, the medium was discarded and replaced with a fresh medium (final DMSO concentration: 0.8%) containing 0-150 μM nimesulide (NIM), NIM-Tetra, HA tetrasaccharide, or NIM-HA disaccharide (NIM-Di) conjugates, and then the plates were incubated for 24 hours. Then, the medium was discarded, and 100 μL medium with 10% MTT solution (MTT stock: 5 mg/mL) was added and then the plates were incubated at 37° C. in the dark for 4 hours. Thereafter, the medium was discarded carefully, and 200 μL of DMSO was added and the plates were shaken for 10 minutes before being read plate at 570 nm.

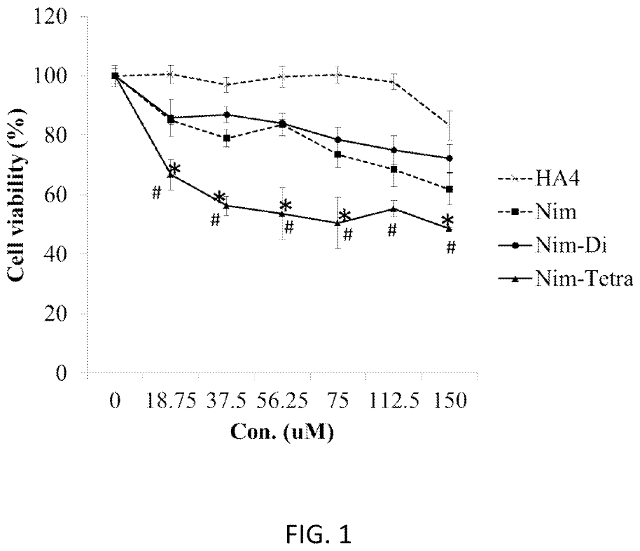

The results, as summarized in FIG. 1, indicate that NIM-Tetra significantly exhibits better tumor cell cytotoxicity to MDA-MB-231 cells, compared with nimesulide alone, HA tetrasaccharide alone, or NIM-HA disaccharide (NIM-Di) conjugates. NIM-HA disaccharide (NIM-Di) also showed comparable tumor cell cytotoxicity activity with Nim.

### Example 3

Tumor Cell Cytotoxicity of NIM-HA Conjugates to A549 Cells

In this example, A549 cells (human lung cancer cell line) were used to assess the tumor cell cytotoxicity of various NIM-HA conjugates.

A549 cells in 100 μL F-12K medium were seeded in a density of 3×10³ per well in a 96-well plate and incubated overnight. The next day, the medium was discarded and replaced with a fresh medium (final DMSO concentration: 0.8%) containing 0-150 μM nimesulide, NIM-Tetra, HA tetrasaccharide, or NIM-HA disaccharide (NIM-Di) conjugates, and then the plates were incubated for 24 hours. Then, the medium was discarded, and 100 μL medium with 10% MTT solution (MTT stock: 5 mg/mL) was added and then the plates were incubated at 37° C. in the dark for 4 hours. Thereafter, the medium was discarded carefully, and 200 μL of DMSO was added and the plates were shaken for 10 minutes before being read plate at 570 nm.

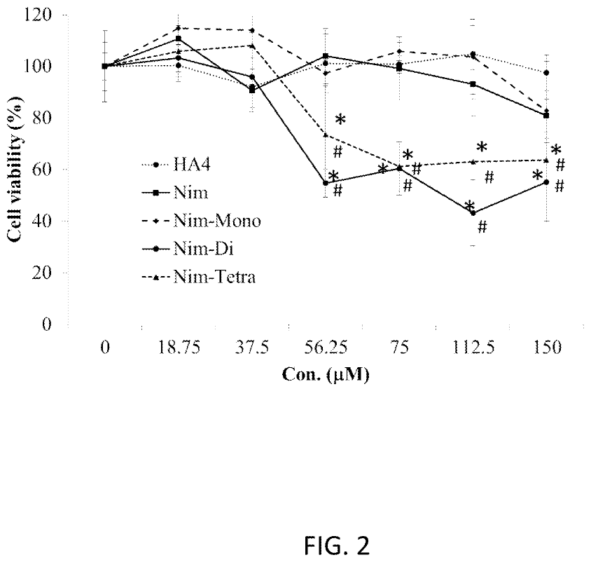

The results, as summarized in FIG. 2, indicate that both NIM-Tetra and NIM-HA disaccharide (NIM-Di) conjugates exhibit better tumor cell cytotoxicity to A549 cells significantly, compared with nimesulide alone or HA tetrasaccharide alone. NIM-HA monosaccharide (NIM-Mono) also showed comparable tumor cell cytotoxicity activity with Nim. NIM-HA disaccharide (NIM-Di) also showed better tumor cell cytotoxicity activity than nimesulide.

### Example 4

Tumor Cell Cytotoxicity of NIM-HA Conjugates to HT-29 Cells

In this example, HT-29 cells (human colorectal cancer cell line) were used to assess the tumor cell cytotoxicity of various NIM-HA conjugates.

HT-29 cells in 100 μL MyCoy's-5 medium were seeded in a density of 3×10³ per well in a 96-well plate and incubated overnight. The next day, the medium was discarded and replaced with a fresh medium (final DMSO concentration: 0.8%) containing 0-150 μM nimesulide (NIM), NIM-Tetra, or HA tetrasaccharide, and then the plates were incubated for 24 hours. Then, the medium was discarded, and 100 μL medium with 10% MTT solution (MTT stock: 5 mg/mL) was added and then the plates were incubated at 37° C. in the dark for 4 hours. Thereafter, the medium was discarded carefully, and 200 μL of DMSO was added and the plates were shaken for 10 minutes before being read plate at 570 nm.

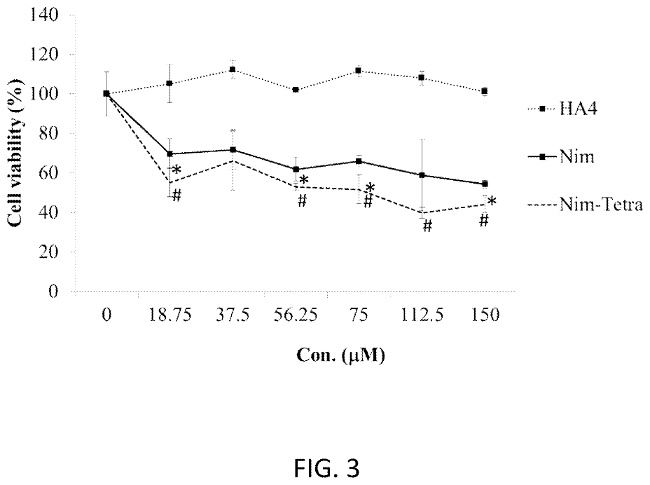

The results, as summarized in FIG. 3, indicate that NIM-Tetra exhibits better tumor cell cytotoxicity to HT-29 cells significantly, compared with nimesulide alone or HA tetrasaccharide alone.

In sum, the results provided in Examples 2 to 4 of the present disclosure show that the present NIM-Tetra conjugate exhibits desired tumor cells cytotoxicity to several cancers, such as breast cancer, lung cancer, and colorectal cancer. NIM-HA disaccharide (NIM-Di) exhibits desired cells cytotoxicity in several cancers, such as breast cancer, lung cancer, and colorectal cancer.

It will be understood that the above description of embodiments is given by way of example only and that those may make various modifications with ordinary skill in the art. The above specification, examples, and data provide a complete description of the structure and use of exemplary embodiments of the invention. Although various embodiments of the invention have been described above with a certain degree of particularity or with reference to one or more individual embodiments, those with ordinary skill in the art could make numerous alterations to the disclosed embodiments without departing from the spirit or scope of this invention.

1. A method for treating cancer in a subject in need thereof, comprising the step of administering to the subject an effective amount of a hyaluronan conjugate, wherein the hyaluronan conjugate comprises one to four disaccharide units of D-glucuronic acid and N-acetylglucosamine, and one or more hydrogenated nimesulide, each covalently linked to the D-glucuronic acid of one of the disaccharide units.

2. The method according to claim 1, wherein the hyaluronan conjugate has the structure of, 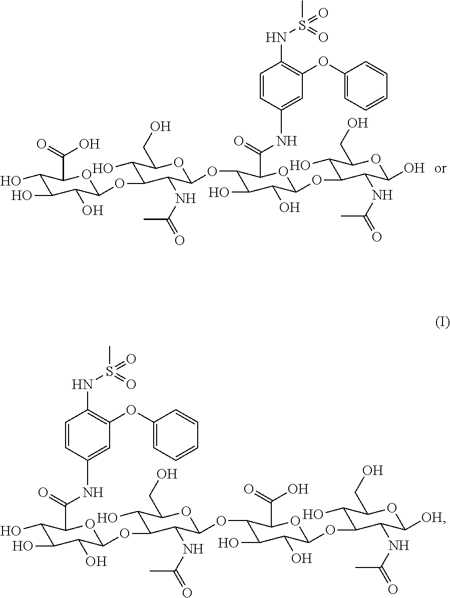 [SMILES: CC(=O)NC1C(O)OC(CO)C(O)C1OC1OC(C(=O)Nc2ccc(NS(C)(=O)=O)c(Oc3ccccc3)c2)C(OC2OC(CO)C(O)C(OC3OC(C(=O)O)C(O)C(O)C3O)C2NC(C)=O)C(O)C1O ; CC(=O)NC1C(O)OC(CO)C(O)C1OC1OC(C(=O)O)C(OC2OC(CO)C(O)C(OC3OC(C(=O)Nc4ccc(NS(C)(=O)=O)c(Oc5ccccc5)c4)C(O)C(O)C3O)C2NC(C)=O)C(O)C1O]
a pharmaceutically acceptable salt thereof.

3. The method according to claim 1, wherein the hyaluronan conjugate has the structure of, 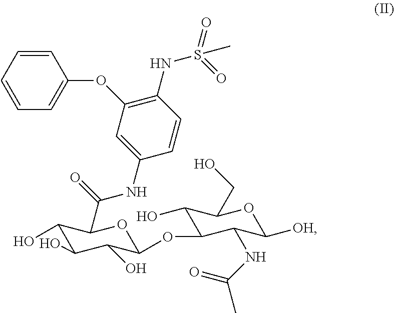 [SMILES: CC(=O)NC1C(O)OC(CO)C(O)C1OC1OC(C(=O)Nc2ccc(NS(C)(=O)=O)c(Oc3ccccc3)c2)C(O)C(O)C1O]
or a pharmaceutically acceptable salt thereof.

4. The method according to claim 1, wherein the hyaluronan conjugate has the structure of, 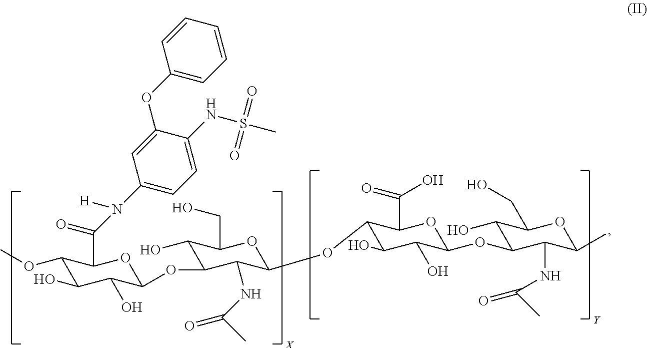
or a pharmaceutically acceptable salt thereof, where X is 1 and Y is 0, 1, 2, or 3.

5. The method according to claim 1, wherein the subject is a human.

6. The method according to claim 1, wherein the cancer is breast cancer, lung cancer, or colorectal cancer.

7. The method according to claim 1, wherein the degree of substitution of the hyaluronan conjugate is 20 to 100%.

8-28. (canceled)
